(19) United States
(12) Patent Application Publication
(10) Pub. No.: US 2004/0122468 A1
(43) Pub. Date: Jun. 24, 2004

(54) Braided intraluminal device for stroke prevention

(75) Inventors: Ofer Yodfat, Modi'in (IL); Boaz Nishri, Doar Na Menashe (IL); Ygael Grad, Tel Aviv (IL); Avraham Rapaport, Tel Aviv (IL); Orna Oz, Herzlia (IL); Yaron Assaf, Doar Na Menashe (IL); Dagan Harris, Hadera (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: MindGuard Ltd.

(21) Appl. No.: 10/724,144

(22) Filed: Dec. 1, 2003

Related U.S. Application Data
(60) Provisional application No. 60/429,551, filed on Nov. 29, 2002.

(51) Int. Cl. A61M 29/00

(52) U.S. Cl. 606/200

(57) ABSTRACT

A diverting filter for implantation in the bifurcation of the human common carotid artery (CCA) with the external carotid artery (ECA) and the internal carotid artery (ICA), comprising: a tubular body expandable from an initial small-diameter state for manipulation through the CCA to an expanded larger-diameter state for implantation in said bifurcation, the tubular body having an at rest state wherein the tubular body exhibits a diameter greater than the expanded larger-diameter state; the tubular body including a proximal region for implantation in the CCA, a distal region for implantation in the ECA, and a middle filtering region for alignment with the orifice of the ICA for diverting relatively-large emboli in the CCA blood flow to the ECA while minimizing interference to blood flow through both the ICA and the ECA; constituted of between 48 and 56 braided filaments exhibiting an average porosity index of at least 80%.

### RELATIONSHIP TO EXISTING APPLICATIONS

This application claims priority from Provisional Patent Application S/No. 60/429,551 filed Nov. 29, 2002, whose entire contents are incorporated herein by reference.

### BACKGROUND OF THE INVENTION

The invention relates generally to the field of implantable intraluminal devices and more particularly to a braided intraluminal device for stroke prevention.

A major portion of blood supply to the brain hemispheres is by two arteries, referred to as common carotid arteries (CCA), each of which bifurcates into an internal carotid artery (ICA) and an external carotid artery (ECA). Blood to the brain stem is supplied by two vertebral arteries.

A stroke is denoted by an abrupt impairment of brain function caused by pathologic changes occurring in blood vessels. The main cause of stroke is insufficient blood flow to the brain (referred to as “an ischemic stroke”), which occurs in about 80% of stroke cases. Ischemic strokes are caused by sudden occlusion of an artery supplying blood to the brain. Occlusion or partial occlusion (stenosis) is typically the result of diseases of the arterial wall. Arterial atherosclerosis is by far the most common arterial disorder, and when complicated by thrombosis or embolism it is the most frequent cause of cerebral ischemia and infarction, eventually causing cerebral stroke.

Cardioembolism causes about 15%-20% of all strokes. Stroke caused by heart disease is primarily due to embolism of thrombotic material forming on the atrial or ventricular wall or the left heart valves. These thrombi then detach and embolize into the arterial circulation. Emboli of a sufficient size can occlude large arteries in the brain territory and cause strokes.

Cardiogenetic cerebral embolism is presumed to have occurred when cardiac arrhythmia or structural abnormalities are found or known to be present. The most common causes of cardioembolic stroke are nonrheumatic (non-valvular) atrial fibrillation (AF), prothestic valves, rheumatic heart disease (RHD), ischemic cardiomyopathy, congestive heart failure, myocardial infarction, port-operatory state and protruding aortic arch atheroma (A.A.A.).

Such disorders are currently treated in different ways such as by drug management, surgery (carotid endarterectomy) in case of occlusive disease, or carotid angioplasty and carotid stents. Endarterectomy, angioplasty and carotid stenting are procedures targeting at opening the occluded artery, however they do not prevent progression of new plaque. Even more so, the above treatment methods only provide a solution to localized problems and do not prevent proximal embolic sources, i.e. an embolus formed at remote sites (heart and ascending aorta), from passing through the reopened stenosis in the carotid and occluding smaller arteries in the brain. It will also be appreciated that endarterectomy is not suitable for intracranial arteries or those in the vertebrobasilar system, since these arteries are positioned within an unacceptable environment (brain tissue, bone tissue) or are too small in diameter.

Introducing filtering means into blood vessels, in particular into veins, has been known for some time. However, filtering devices known in the art are generally of a complex design, which renders such devices unsuitable for implantation within carotid arteries, and unsuitable for handling fine embolic material. However, when considering the possible cerebral effects of even fine embolic material occluding an artery supplying blood to the brain, the consequences may be fatal or may cause irreversible brain damage. There is therefore significant importance to providing suitable means for preventing even small embolic material from entering the internal carotid artery, so as to avoid brain damage.

A further drawback of prior art filtering means is their tendency to become clogged. On the one hand, in order to provide efficient filtering means, the filter should be of fine mesh. On the other hand, a fine mesh has a higher tendency toward, and risk of, occlusion. It should also be noted that the flow ratio between the ICA and the ECA is about 4:1. This ratio also reflects the much higher risk of embolic material flowing into the ICA.

The average porosity index (PI) of a mesh like tubular implantable device is defined by the relation:

$$PI = 1 - \frac{Sm}{St}$$

wherein: “Sm” is the actual surface covered by the mesh-like tube , and “St” is the total surface area of the mesh-like tube.

U.S. Pat. No. 6,348,063 entitled “IMPLANTABLE STROKE TREATING DEVICE” issued to Yassour et al., and U.S. Published patent application Ser. No. 2003/0,125,801 entitled “IMPLANTABLE STROKE TREATING DEVICE”, the contents of both being herein incorporated by reference, describe a method and a device for preventing the embolic material flowing in the CCA from accessing the ICA, comprising deflecting the flow of said embolic material into the ECA without blocking the ICA. A number of solutions are described, leading to many possible combinations of elements that may be used.

U.S. patent application Ser. No. 10/311,876 entitled “IMPLANTABLE BRAIDED STROKE PREVENTING DEVICE AND METHOD OF MANUFACTURING” filed Dec. 20, 2002 and published as International application WO 02/05729, listing Yodfat et al. as inventors, whose contents are incorporated herein by reference, describes an implantable deflecting device for positioning in the vicinity of an arterial bifurcation comprising a braided tubular body. A number of solutions are described, leading to many possible combinations of filament sizes and number of filaments, porosity index, and length of a side of its openings.

An object of the present invention is to provide a diverting filter for implantation in the bifurcation of the human CCA with the ECA and the ICA, having specific and critical design characteristics that will maximize the deflection of embolic material to the ECA, while minimizing interference to the blood flow through the ICA and the occlusion of the diverting filter by embolic material or neointimal growth.

### SUMMARY OF THE INVENTION

Accordingly, it is a principal object of the present invention to overcome the disadvantages of prior art by providing specific design and critical design characteristics that will optimally deflect embolic material to the ECA, while minimally interfering with blood flow to the ICA and prevent the occlusion of the diverting filter by embolic material or neointimal growth.

This is provided in the present invention by a diverting filter for implantation in the bifurcation of the human CCA with the ECA and the ICA, comprising: a tubular body expandable from an initial small-diameter state for manipulation through the CCA to an expanded larger-diameter state for implantation in the bifurcation; the tubular body including a proximal region for implantation in the CCA, a distal region for implantation in the ECA, and a middle filtering region for alignment with the orifice of the ICA for diverting relatively-large emboli in the CCA blood flow to the ECA while minimizing interference to blood flow through both the ICA and the ECA; the tubular body being constituted of between 48 and 56 braided filaments each having an outer diameter of 48-52 μm and braided into a tubular body exhibiting an average porosity index of at least 80% when in the expanded state.

In a preferred embodiment the average porosity index of the diverting filter is 80-83%. In another preferred embodiment the tubular body exhibits an at rest state wherein the tubular body exhibits a diameter greater than the expanded larger-diameter state. In the at rest state of the tubular body the distal region has an outer diameter gradually decreasing from the middle filtering region and terminating in an outwardly flared distal end, and the proximal region has an outer diameter gradually increasing from the middle filtering region and terminating in an outwardly flared proximal end. In one further preferred embodiment the outer diameter of the outwardly flared distal end is increased by more than 0.4 mm in respect to the distal region. In another further preferred embodiment the outer diameter of the outwardly flared proximal end is increased by more than 0.2 mm in respect to the proximal region. In yet another further preferred embodiment, in the at rest state of the tubular body, the outer diameter of the distal region is 7.3-7.7 mm. In yet another further preferred embodiment, in the at rest state of the tubular body, the outer diameter of an end of the distal region is 7.8-8.6 mm. In yet another further preferred embodiment, in the at rest state of the tubular body, the outer diameter of the proximal region is 7.7-8.1 mm. In yet another further preferred embodiment, in the at rest state of the tubular body, the outer diameter of an end of the proximal region is 8.1-8.5 mm. In yet another further preferred embodiment the outer diameter of the outwardly flared distal end is increased by more than 0.4 mm, and the outer diameter of the outwardly flared proximal end is increased by more than 0.2 mm. In yet another further preferred embodiment the length of the tubular body in the at rest state is 30-34 mm.

In one embodiment the tubular body is constituted of one of 48 and 56 of the braided filaments. In another embodiment the average porosity index in the middle region is defined by windows having an inscribed diameter of 400-500 μm in the expanded larger-diameter state. In yet another embodiment the average porosity index in the middle region is defined by windows having an inscribed diameter of 450-500 μm in the expanded larger-diameter state.

The invention also provides for a diverting filter for implantation in the bifurcation of the human CCA with the ECA and the ICA, comprising: a tubular body expandable from an initial small-diameter state for manipulation through the CCA to an expanded larger-diameter state for implantation in the bifurcation; the tubular body including a proximal region for implantation in the CCA, a distal region for implantation in the ECA, and a middle filtering region for alignment with the orifice of the ICA for diverting relatively-large emboli in the CCA blood flow to the ECA while minimizing interference to blood flow through both the ICA and the ECA; the tubular body being constituted of a plurality of braided filaments each having an outer diameter of 48-52 μm and braided into a tubular body exhibiting an average implanted braid angle of 70°-110° in the middle filtering region when in the expanded larger-diameter state.

In one exemplary embodiment the diverting filter exhibits an average implanted braid angle of 70°-105° in the middle filtering region when in the expanded state. In another exemplary embodiment the diverting filter exhibits an average implanted braid angle of 80°-100° in the middle filtering region when in the expanded state.

In one embodiment the plurality of braided filaments is between 48 and 56 braided filaments. In another embodiment the plurality of braided filaments is one of 48 and 56 braided filaments.

The invention also provides for a diverting filter for implantation in the bifurcation of the human CCA with the ECA and the ICA, comprising: a tubular body expandable from an initial small-diameter state for manipulation through the CCA to an expanded larger-diameter state for implantation in the bifurcation; the tubular body including a proximal region for implantation in the CCA, a distal region for implantation in the ECA, and a middle filtering region for alignment with the orifice of the ICA for diverting relatively-large emboli in the CCA blood flow to the ECA while minimizing interference to blood flow through both the ICA and the ECA; the tubular body being constituted of a plurality of braided filaments braided into a tubular body exhibiting an inscribed diameter of 400-500 μm in the middle filtering region when in the expanded state.

In one embodiment the diverting filter exhibits an inscribed diameter of 450-500 μm in the middle filtering region when in the expanded state. In another embodiment the middle filtering region exhibits an average implanted braid angle of 75°-105° in the middle filtering region when in the expanded state. In yet another embodiment the plurality of braided filaments is between 48 and 56 braided filaments. In one further embodiment the plurality of braided filaments constitute filaments each having an outer diameter of between 48-52 um. In another embodiment the plurality of braided filaments constitute filaments each having an outer diameter of between 48-52 um.

Additional features and advantages of the invention will become apparent from the following drawings and description.

### BRIEF DESCRIPTION OF THE DRAWINGS

For a better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings, in which like numerals designate corresponding elements or sections throughout and in which:

FIG. 1 illustrates a schematic illustration of a typical human carotid artery;

FIG. 2 illustrates an implanted diverting filter in accordance with the principle of the present invention;

FIGS. 3a-3c illustrates a diverting filter in accordance with the principle of the present invention in its initial small diameter state expanding to a larger-diameter state;

FIG. 4 illustrates another expanded view of a portion of the diverting filter of FIG. 2;

FIG. 5 illustrates Neointimial (NI) coverage percentage versus percentage of implantations for each of three types of diverting filters;

FIGS. 6a-6c illustrates NI coverage percentage versus number of implantations after 2-4 weeks, 10-13 weeks and 16-18 weeks follow up, respectively;

FIG. 6d illustrates the direction of NI growth;

FIG. 7 illustrates the percentage of opening of the distal and proximal edges for each of three types of diverting filters;

FIGS. 8a-8c illustrates NI coverage percentage as a function of inscribed diameter for each of three diverting filter types, respectively;

FIGS. 9a-9c illustrates NI coverage percentage as a function of radial force for each of the diverter types, respectively; and

FIG. 10 illustrates a diverting filter designed in accordance with the principle of the current invention.

### DETAILED DESCRIPTION OF THE PREFERRED EMBODIMENTS

The present embodiments enable a diverting filter for implantation in the bifurcation of the human common carotid artery (CCA) with the external carotid artery (ECA) and the internal carotid artery (ICA) having specific design characteristics that will not be occluded in the patient body by emboli or neointimal growth and providing an average porosity index (PI) of at least 80% in the diverting filter region.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

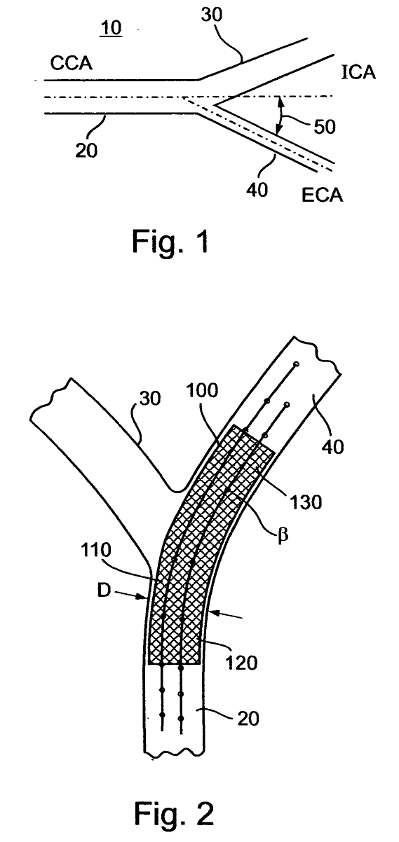

FIG. 1 schematically illustrates a typical human carotid artery 10 showing the bifurcation of the CCA 20 into the ICA 30 and the ECA 40 and the angle 50 between the longitudinal axes of CCA 20 and ECA 40. Table I illustrates typical average diameters in mm of CCA 20, ICA 30, ECA 40 and the calculated ECA/CCA diameter ratio based on the medical literature.

TABLE I

| | ENTIRE | | |
|---|---|---|---|
| Diameter | GROUP | MALE | FEMALE |
| CCA (mm) | ˜7.2 | ˜7.8 | ˜6.8 |
| ICA (mm) | ˜5.2 | ˜5.3 | ˜4.3 |
| ECA (mm) | ˜4.7 | ˜4.9 | ˜4.1 |
| ECA/CCA | ˜0.6 | ˜0.6 | ˜0.6 |
| ratio | | | |

Angle 50 is formed by the longitudinal axes of CCA 20 and ECA 40, and is quite variable among the population. Angle 50 has been calculated to be between 10° and 48°, however due to the wide variability it is recommended that a range of 0° and 70° be designed for.

Table II represents an overall summary of the anatomical range found in the literature, indicating the mean, minimum and maximum diameter and the angle, respectively, of CCA 20, ICA 30, ECA 40 and angle 50, in addition to the typical lengths expressed in mm found for CCA 20, ICA 30 and ECA 40.

TABLE II

| | Mean | Minimum | Maximum | |
|---|---|---|---|---|
| | Value | Value | Value | Length |
| CCA 20 | 7.2 mm | 5.0 mm | 11.0 mm | Right 13 ± 4 mm |
| | | | | Left 16 ± 4 mm |
| ICA 30 | 5.2 mm | 3.5 mm | 10.4 mm | 15 ± 2.5 mm |
| ECA 40 | 4.7 mm | 3.1 mm | 9.8 mm | 15 ± 2.5 mm |
| Angle 50 | 25° | 10° | 100° | |

FIG. 2 illustrates diverting filter 100 in accordance with the principle of the subject invention, dimensioned and configured to be implanted in the human distal CCA 20 to the proximal ECA 40 with its mid-region facing the orifice of ICA 30. The intended clinical use of the filter of the subject invention is in long-term prevention of embolic stroke. The filtering part of the diverting filter of the subject invention has the role of preventing proximally originating emboli from penetrating into ICA 30 by rerouting them to ECA 40, while maintaining the blood flow to the intra-cranial vascular bed through ICA 30. Diverting filter 100 exhibits a proximal region 120 implanted within CCA 20, a distal region 130 implanted within ECA 40, a middle region 110 covering the opening of ICA 30 and diameter D, defined at each point along the length of diverting filter 100 by the inner wall of the blood vessel in which diverting filter 100 is implanted.

The geometry of diverting filter 100 of the subject invention is a generally tubular shaped braided wire mesh as illustrated in FIG. 2, with the following requirements: filtering particles larger than the predetermined value of 500 μm; presenting minimized blood flow disturbance in terms of both local and global flow; biocompatible; radio-opaque; non-invasively implanted; self expandable; rigid enough to be deployed and anchored in the artery; flexible enough to enable appositioning to the tapered vessel wall and ultimate fixation; and good mechanical failure resistance. The design also considers the following issues and their interaction: biological response; clinical and procedural demands; geometrical behavior and mechanical and material behavior. It is an important design criteria that the ICA not be blocked by emboli or neointimal growth, as such blockage will lead to negative patient outcome.

From a hemocompatibility and hemodynamic point of view a foreign object in contact with arterial blood flow may activate the coagulation system. The resulting flow induced thrombogenicity is primary due to platelet activation. According to the literature, the hemodynamic parameters, which activate the coagulation system comprise the following: high shear rate; low shear rate; long residence time and regions of recirculation and flow stagnation. The main idea is to reach creeping flow conditions (also known as Stockes' flow), with Re<4 (Re=Ud/ν, where U is the blood velocity, d is the round filament diameter or another cross section characteristic length, and ν is the dynamic viscosity).

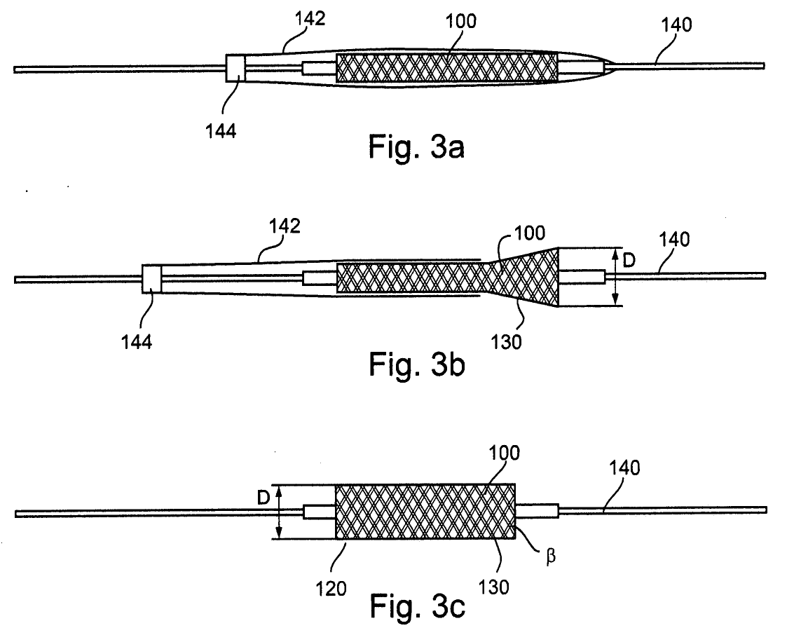

FIGS. 3a-3c illustrate diverting filter 100 in various stages of deployment from a small diameter state for manipulation through CCA 20 to an expanded larger-diameter state for implantation in the bifurcation of the ECA 40 and ICA 30. FIG. 3a illustrates diverting filter 100 restrained to a small diameter state by sheath 142 and being directed to the implantation location along guidewire 140. Restraining ring 144 functions to allow withdrawal of sheath 142. FIG. 3b illustrates diverting filter 100 being partially deployed as sheath 142 is withdrawn proximally from diverting filter 100. Distal region 130 of diverting filter 100 expands to artery diameter D, thus securing itself in place. FIG. 3c illustrates diverting filter 100 being fully released from sheath 142, both distal region 130 and proximal region 120 are now fully expanded to a larger-diameter state, generally exhibiting diameter D of the artery wall. It is to be understood that the value of diameter D is not uniform over the length of implanted diverting filter 100, and closely follows the inner wall diameter of CCA 20 in proximal region 120 and ECA 40 in distal region 130.

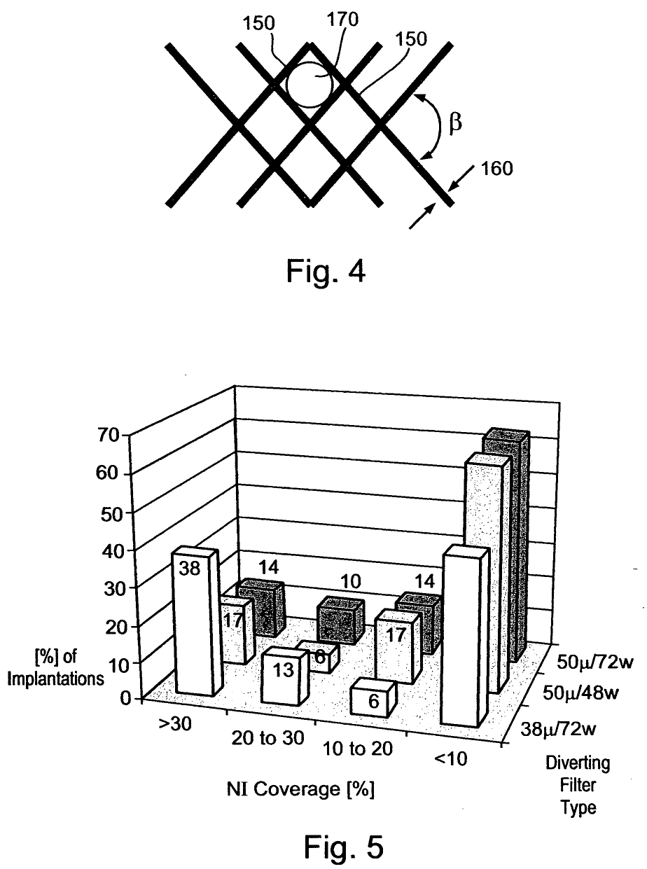

FIG. 4 illustrates an enlarged portion of the diverting filter 100 of FIG. 2 showing filaments 150 exhibiting a characteristic diameter 160, filaments 150 being braided at a braid angle β defining typically diamond shaped openings, hereinafter called windows, having an inscribed diameter 170. Inscribed diameter 170 is also interchangeably referred to hereinafter, as Dᵢ. The tubular shaped wire mesh diverting filter 100 of the present invention comprises a braid of substantially uniform filaments 150 braided at a braid angle β. Diverting filter 100 of the subject invention achieves the above requirements by utilizing a reduced filament diameter 160 (or another cross section characteristic length) as much as possible within the structural strength demands. It is evident that the higher the PI, and the smaller the filament diameter 160, the less the disturbance of the diverting filter of the subject invention to the blood flow. The high PI of a mesh having a given braid angle β can be achieved in two ways: (a) by increasing the gap between the filaments 150, thus increasing inscribed diameter 170, or (b) by decreasing the filament diameter 160 (or another cross section characteristic length).

Another biological aspect that should be taken into consideration to ensure that the mesh remains open when implanted in the artery is neointimal growth (NIG). High local shear stress tends to restrain NIG. In general, the local shear stress on a filament depends on the local curvature with τ˜1/c, where τ is the shear stress and “c” is defined as the curvature of filament 150. Diverting filter 100 of the subject invention is configured with a small filament diameter 160 (or another cross section characteristic length) in order to minimize NIG and the risk for filter blockage. NIG also depends on inscribed diameter 170, PI and braid angle β in a way that is not completely clear. According to literature on the subject of aneurysm treatment by stenting, the PI threshold for the mesh to occlude is around 70%. However, it would be reasonable to assume that PI is not a unique geometrical parameter characterizing NIG. It is evident from asymptotic analysis that a mesh with a very small Dᵢ will be occluded by NIG independently of PI. While the threshold is not precisely known, it probably depends on the scale of the blood particles (note that at a small size scale, the blood is not a homogenous fluid, but a suspension), the diffusion scale of the factors that are responsible for connection between the intima cells, etc. The window pattern is dependent on PI, Dᵢ and filament diameter 160.

In summary, from the hemocompatibility and hemodynamics point of view, a small filament diameter 160 (or another cross section characteristic length) mesh is the best solution for the diverting filter of the subject invention. The advantages of such an approach are as follows: reduction of wake, including swirls, vortices and re-circulation regions; reduction of platelet residence time in the proximity of the filament 150; possibility to decrease Dᵢ while still maintaining a high PI, and increase of the local shear stress thus decreasing NIG.

### Mechanical and Geometrical Analysis

From a mechanical and geometrical point of view, appositioning and coupling of proximal region 120 to CCA 20 and of distal region 130 to ECA 40 as shown in FIG. 2 are important for a long-term proper functioning of the diverting filter of the subject invention. In the prior art development of endovascular stents the main goal has been to produce radial pressure and rigidity sufficient to expand and lend support to the vessel with a minimal injury to the vessel wall. Contrastingly, in the development of the diverting filter of the subject invention, the main goal is to achieve a minimum Dᵢ (170 of FIG. 4), minimal blood flow disturbance (local and global) while maintaining sufficient radial and longitudinal forces and rigidity to position and maintain diverting filter 100 in the vessel. Taking into consideration the hemocompatibity and hemodynamical point of view, critical for the fulfillment of the safety requirements, the geometry and material composition are determined to be very low radial and longitudinal forces and rigidity compared to endovascular stents, as described hereinto below using tubular braided structure analysis.

The tubular braided diverting filter 100 of the subject invention mechanical and geometrical behavior can be described using the following five main equations:

$$\begin{matrix}Di = \frac{2 π D sin ( β )}{N} - d & Equation 1 \\ PI = \frac{Nd π D - \frac{{N}^{2} {d}^{2}}{8 sin ( β )}}{{( π D )}^{2} sin ( β )} & Equation 2 \\ F = \frac{π N {cos}^{2} {β}_{0} {d}^{4}}{16 {D}_{0}^{2}} * ( 2 G \{ \sqrt{1 - {( \frac{D}{{D}_{0}} cos {β}_{0} )}^{2}} - sin {β}_{0} \} - E \{ ( \frac{D}{{D}_{0}} - 1 ) \sqrt{{( \frac{{D}_{0}}{D} )}^{2} - {cos}^{2 } {β}_{0}} \} ) & Equation 3 \\ P a = \frac{N {cos}^{4} {β}_{0} {d}^{4}}{8 {D}_{0}^{4} [ 1 - {( \frac{D}{{D}_{0}} cos {β}_{0} )}^{2} ]} * ( 2 G \{ \sqrt{1 - {( \frac{D}{{D}_{0}} cos {β}_{0} )}^{2}} - sin {β}_{0} \} - E \{ ( \frac{D}{{D}_{0}} - 1 ) \sqrt{{( \frac{{D}_{0}}{D} )}^{2} - {cos}^{2 } {β}_{0}} \} ) & Equation 4 \\ {K}_{p} ≡ \frac{ⅆ P a}{ⅆ D} and {K}_{L} ≡ \frac{ⅆ F}{ⅆ L} , & Equation 5\end{matrix}$$

where:

Dᵢ—window inscribed diameter (170 of FIG. 4)

N—number of filaments

D—artery diameter

L—the length of diverting filter 100 of FIGS. 2-4

PI—porosity index

d—filament diameter (160 of FIG. 4)

Pa—average radial pressure

K_{P}—radial rigidity

K_{L}—longitudinal rigidity

β—braid angle

E—modulus of elasticity

G—modulus of rigidity

‘0’ subscript defines values of diverting filter 100 on a braiding mandrel.

Assuming a uniform braiding angle on a braiding mandrel β₀, an implanted braid angle β, and a round filament made of a pre-determined uniform material, the following relations for diverting filter 100 in the implanted state are valid:

Dᵢ˜D/N(for D/N>>d) Equation 6

PI˜Dᵢ/d˜1/N(D/d) Equation 7

Pa˜N(d/D)⁴ Equation 8

K_{P}˜Nd⁴/D⁵ Equation 9

K_{L}˜Nd⁴/D². Equation 10

In order to filter particles as small as possible, while preserving a high PI, it is desirable based on Equation 7 to decrease filament diameter 160 (FIG. 4) as much as possible. Unfortunately, decreasing filament diameter 160 leads to a sharp drop in general mechanical properties, including: radial pressure, radial and longitudinal rigidity (equations 8 to 10). The mechanical properties also depend on the number of filaments ‘N’, as well as on the diameter of the artery, ‘D’, in which the intraluminal device is to be implanted. The number of filaments having a given diameter, d, defines Dᵢ for a given D, and changes only slightly with a change in the filament and artery diameters. The number of filaments, N, influences the mechanical properties linearly (equations 7 to 10), and it is evident that it is only a secondary parameter considering the mechanical behavior. The braiding angle β₀, the modulus of elasticity of the filament material and the modulus of rigidity of the filament material influence the mechanical properties (equations 3 to 4), but similar to N, only in a moderate manner.

The above analysis leads to the conclusion that once artery diameter D and number of filaments N are determined, the possibility to increase the mechanical properties is limited mainly because of the biological constraints on filament diameter 160, also interchangeably referred to as filament diameter ‘d’. The above model is based on round cross section filaments and the power of 4 in the filament diameter ‘d’ refers to I and Ip (the moment of inertia and polar moment of inertia of the filament, respectively).

It is important to note that there is no requirement for a large radial force, as the function of the device is to filter and divert emboli. Therefore, the structure can be delicate, as long as it remains sufficiently rigid to apposition itself in the artery without migration thus ensuring ultimate fixation. This is in contradistinction to stents of the prior art, whose primary function is to exhibit a large radial force so as to support a weakened blood vessel.

Braid angle β changes as the diameter of diverting filter 100 changes. Thus diverting filter 100 exhibits an initial braid angle β₀ on a braiding mandrel, and exhibits a diameter imposed by the braiding mandrel. Braided diverting filter 100 changes diameter and overall length in concert with a change in braid angle β, and thus upon implantation diverting filter 100 exhibits an implanted braid angle β with diverting filter 100 having diameter D defined by the blood vessel in which it is implanted.

In a preferred embodiment, filaments 150 comprise wires of cobalt based alloy type (ASTM F 1058, Grade 2) due to its good corrosion resistance combined with very high mechanical properties, good resistance to fatigue and wear and sufficient ductility to enable the braiding process. Furthermore, the low percentage of Beryllium is advantageous from a biocompatibility standpoint. Preferably, a 45-48% cold reduction after final annealing is accomplished to ensure optimal mechanical spring properties. Preferably filament 150 of diverting filter 100 comprise round wires exhibiting a diameter between 48-52 μm.

### Fatigue and Stress Analysis

A fatigue and stress Finite Element Analysis (FEA) was performed on various constructs of diverting filter element 100 to obtain an estimation and prediction of service life. An analysis was made of both a diverting filter 100 consituted of a plurality of 30 μm round wire filaments and 50 μm wire filaments. Fatigue estimation is made on the basis of the data obtained by relevant tests (e.g. Rotating beam U-bend spin test).

Two constitutive models of the carotid material and two types of wire-to-wall compliance in the region of the artery bifurcation are considered. Two types of material description are commonly used in solid mechanics. The first type assumes the existence of a direct functional dependence between stress and strain. In the simplest case of linear dependence it gives an extension of Hooke's law to the three dimensional state. For an isotropic material two material constants are required, e.g. modulus of elasticity (E) and shear modulus (G). The nonlinear behavior is caused by geometric nonlinearity only, i.e. the body is undergoing large displacements, but the strain remains relatively small (up to 5%-7%). The first material model is based on the previous analyses and presents a simple elastic material with the defined Young modulus and Poisson coefficient, which have been extracted from experimentally observed behavior of a silicon tube (silicon RTV 615). The second material model is based upon the constitutive equation developed by A. Delfino and uses the hyperelastic material capabilities of MSC.NASTRAN. The behavior of such a model is established by assuming the existence of a function, which defines the strain energy stored in material during deformation in terms of strain and material constants. This approach is general and includes simple elastic materials.

In each of these two material models the filament wires are connected to the wall at two different places. The first connection is along the bifurcation window circumference. In the second type of connection the points of filament wire-to-wall connections are shifted inside the bifurcating artery lumen.

A numerical model of diverting filter element 100 was subjected to quasistatic pulsating pressure with the following minimum and maximum values:

Pₘᵢₙ=80 mm Hg

Pₘₐₓ=120 mm Hg.

With minimum pressure being taken as the zero level. Hence the alternating stress applied to the inner surface of the artery varies between

Pₘᵢₙ=0 mm Hg

Pₘₐₓ=40 mm Hg.

The artery is modeled by CHEXA elements with eight nodes. This type of element possesses full nonlinear capabilities, i.e. it could be used with hyperelastic materials. The wall is modeled by three elements through the thickness.

Diverting filter 100 at the bifurcation was modeled by CBEAM elements. This type of element presents a beam possessing capabilities of very large displacements and rotations. A very significant property of this element, which should be noted, is the full coupling between axial and lateral forces in case of nonlinear analysis. The CBEAM elements are connected rigidly to the CHEXA elements with three translational degrees of freedom, which induces interactive forces into the filament wires. This assumption seems to be very conservative, because it does not take into account the relative sliding between the filament wires and the artery. The geometry of diverting filter 100 consists of 48 filament wires of 50 or 30 μm wire diameter, while the outer diameter of diverting filter 100 is 8 mm.

The model consists of 11520 CHEXA, 272 CBEAM elements and 15308 nodes. Because of nonlinearity the problem was solved by increments using a Newton-Raphson iterative procedure at each step of loading. The full loading path was divided into four increments corresponding to the pressure increment by 10 mm Hg at each step. Each increment 10 subdivisions (sub-increments) were taken to provide accuracy and stability of the numerical process.

Each increment of 10 mm Hg is taken as 100%, so that the outputs are presented for 100%, 200%, 300% and 400% corresponding to 10 mm Hg, 20 mm Hg, 30 mm Hg and 40 mm Hg. Maximum stresses in the beam elements during the cycle of loading, the amplitude and the mean stress of the cycle for the two considered types of filament wire-to-artery connections and different filament wire diameters are summarized in Table III and Table IV below in which the stress is expressed in MPa. As can be observed the pressure-stress relation is not linear. The amplitude of the cyclic stress is half of the maximum range, while the mean stress of the cycle is the value of the stresses at the pressure of 20 mm Hg.

TABLE III

| | Filament Wire Diameter | | | |
|---|---|---|---|---|
| | 50 μm | | 30 μm | |
| | Type of Artery Model | | | |
| | Equivalent | | Equivalent | |
| | elastic | Hyperelastic | elastic | Hyperelastic |
| Pressure | | | | |
| [mm Hg] | | | | |
| 10 | 91 | 112 | 188 | 274 |
| 20 | 175 | 212 | 330 | 431 |
| 30 | 251 | 299 | 435 | 575 |
| 40 | 320 | 376 | 519 | 620 |
| Characteristic | | | | |
| stresses of cycle | | | | |
| (MPa) | | | | |
| Amplitude (S_{act}) | 160 | 188 | 259.5 | 310 |
| Mean stress (Sₘ) | 175 | 212 | 330 | 431 |

TABLE IV

| | Filament Wire Diameter | | | |
|---|---|---|---|---|
| | 50 μm | | 30 μm | |
| | Type of Artery Model | | | |
| | Equivalent | | Equivalent | |
| | elastic | Hyperelastic | elastic | Hyperelastic |
| Pressure | | | | |
| [mm Hg] | | | | |
| 10 | 98.5 | 138 | 212 | 310 |
| 20 | 189 | 261 | 375 | 506 |
| 30 | 272 | 370 | 545 | 716 |
| 40 | 352 | 462 | 700 | 898 |
| Characteristic | | | | |
| stresses of cycle | | | | |
| (MPa) | | | | |
| Amplitude (S_{act}) | 176 | 236 | 350 | 449 |
| Mean stress (Sₘ) | 189 | 261 | 375 | 506 |

The essential feature of the problem of calculating a safety factor is that during the cycle of loading-unloading the filament wires do not undergo plastic deformation, and the load is not a reversal, but a pulsating one. In such cases (absence of plastic deformation) the fatigue life estimation for infinitely long time is made on the basis of S-N (stress verus cycles) curve transformed into a Goodman diagram, which presents the actual alternating stress state as a point in coordinates Sₘₐₓ-Sₘᵢₙ. The Goodman diagram can be described by a straight line relation between the maximum allowable amplitude Sₐ, endurance limit for fully reversal stress Sₑ, ultimate stress of material S_{U} and the mean stress of the cycle:

$${S}_{a} = {S}_{e} [ 1 - \frac{{S}_{m}}{{S}_{U}} ] \cdot \qquad Equation 11$$

Now the safety factor against fatigue failure (estimation for infinite life) is made as follows:

$$n = \frac{{S}_{a}}{{S}_{act}} , \qquad Equation 12$$

where S_{act} is the actual amplitude.

One of the critical points is to derive realistic values of the material endurance limit, which takes into account the type of filament wire and the treatment (heat treatment, rolling, etc.). Based on available data, the ultimate tensile strength (40% reduction) S_{U}=281 ksi=1940 Mpa and fatigue strength in reverse bending is 580 Mpa. The safety factors for all the cases considered are summarized in Table V and Table VI below.

TABLE V

| | Filament Wire Diameter | | | |
|---|---|---|---|---|
| | 50 μm | | 30 μm | |
| | Type of Artery Model | | | |
| | Equivalent | | Equivalent | |
| | elastic | Hyperelastic | elastic | Hyperelastic |
| Characteristic | | | | |
| stresses of cycle | | | | |
| (MPa) | | | | |
| Amplitude (S_{act}) | 160 MPa | 188 MPa | 259.5 MPa | 310 MPa |
| Mean stress (Sₘ) | 175 MPa | 212 MPa | 330 MPa | 431 MPa |
| 1 − Sₘ/S_{U} | 0.91 | 0.891 | 0.830 | 0.778 |
| Sₐ | 528 MPa | 517 MPa | 481 MPa | 451 MPa |
| Safety factor | | | | |
| n | 3.30 | 2.75 | 1.85 | 1.45 |

TABLE VI

| | Filament Wire Diameter | | | |
|---|---|---|---|---|
| | 50 μm | | 30 μm | |
| | Type of Artery Model | | | |
| | Equivalent | | Equivalent | |
| | elastic | Hyperelastic | elastic | Hyperelastic |
| Characteristic | | | | |
| stresses of cycle | | | | |
| (MPa) | | | | |
| Amplitude (S_{act}) | 176 MPa | 236 MPa | 350 MPa | 449 MPa |
| Mean stress (Sₘ) | 189 MPa | 261 MPa | 375 MPa | 506 MPa |
| 1 − Sₘ/S_{U} | 0.901 | 0.865 | 0.806 | 0.739 |
| Sₐ | 523 MPa | 502 MPa | 468 MPa | 428 MPa |
| Safety factor | | | | |
| n | 2.97 | 2.12 | 1.33 | 0.95 |

The results of the analyses performed show an increase in the stress level in case the points of connection are shifted into the lumen of the bifurcation window. It may be caused by the shifting of the attachment points, thus leading to an increase of the filament wire arc which in turn leads to increasing the bending moment and stresses, and because shifting of the filament wires into the contour of the bifurcation window increases the rigidity of the attachment points in the model, so that the artery imposes displacements on the ends of the beam elements.

From the performed analyses for different configurations (different types of connection) of diverting filter 100 and two types of artery models it is observed that variation in the filament wire stress value caused by a change in the type of the artery material is in the range between 18% and 35%. It means the equivalent elastic material may be taken as accurate enough for preliminary studying the filter artery interaction behavior, and furthermore in the second type of filament wire-to-artery connection (the points of attachment shifted inward the contour) an increasing stress level is observed. Due to uncertainties in the real state of compliance, this case should be considered as a critical one for fatigue life estimation. A further result is that a significant increase of the filament wire stresses is observed for the second type of filament wire-to-wall connection—up to 44% for 30 μm diameter wire. This leads to decreasing the fatigue safety factors below the desired level.

It is to be noted that the minimum safety factor for the 50 μm diameter wires reaches 2.12, which should be considered as acceptable, whereas the safety factor for the 30 μm diameter wires is low.

### Animal Studies

Several combinations of numbers of filaments and wire sizes were initially analyzed in animal studies to optimize the design of diverting filter 100 of FIG. 2. In particular, the mechanical behavior in terms of compliancy thus minimizing migration from the initial implanted location, as well the biological compatibility in terms of patency for maintained filtered blood flow and NIG were analyzed.

Average PI of approximately 80% was maintained in all samples by changing the number of filaments N in combination with filament diameter 160, and diameter D of the implanted filter. The devices were implanted in the external iliac to the external femoral of female swine, thus filtering the internal femoral artery. This implantation site was chosen on the basis of the assumption that it presents a good simulation of the anatomy of human common carotid bifurcation, though different hemodynamically. Indeed, the blood flow through these arteries is lower, compared to human common carotid circulation. These hemodynamical conditions in concomitance with the fact that the iliac arteries are muscular arteries, compared to the elastic carotid arteries, can be considered a situation with a higher susceptibility to NIG. These hemodynamical conditions are therefore indicative of a worst case scenario.

Diverting filters comprising 48 filaments of 50 μm round wire (hereinafter 50μ/48 w), 72 filaments of 50 μm round wire (hereinafter 5082 /72 w) and 72 filaments of 38 μm round wire (hereinafter 38μ/72 w) were tested and a complementary data analysis, mainly focusing on the mechanical behavior of the implanted diverting filter and the neointimal (NI) coverage as a function of the mechanical design was performed at up to 4 months follow up. No thrombus formation was observed on the filtering filaments for any of the above designs. NI coverage was estimated using morphometrical software, and is illustrated in FIG. 5, in which the x-axis represents the percentage of NI coverage, the y-axis represents percentage of implantations and the z-axis represents the diverting filter type. Designs with wire diameter of 50μ had less NI coverage, in particular, 76% and 78% of the 50μ/72 w and 50μ/48 w designs, respectively, had less than 20% NI coverage, while only 50% of the 38μ/72 w design displayed this value.

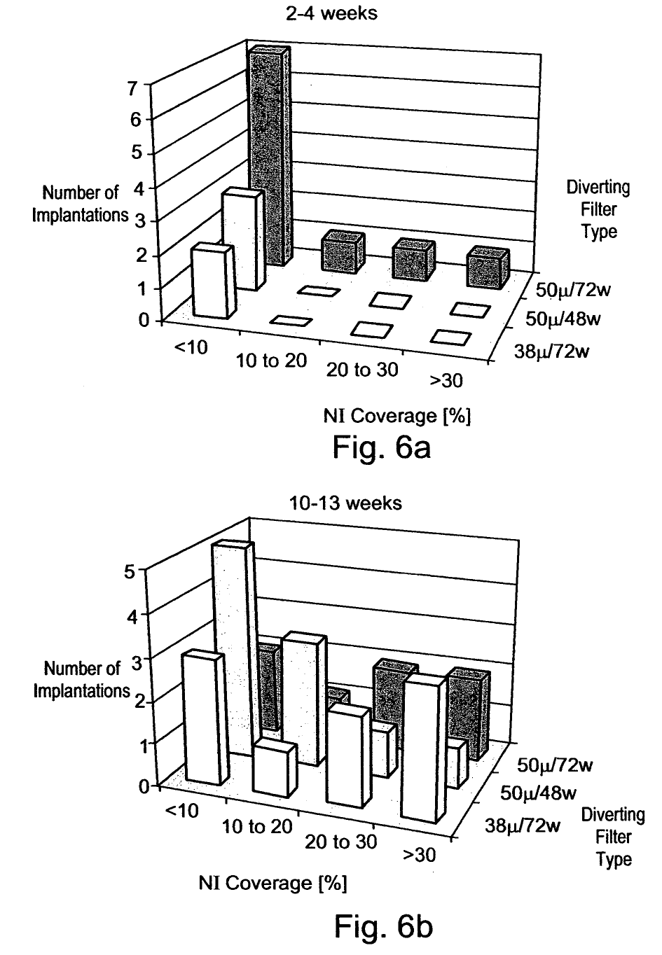

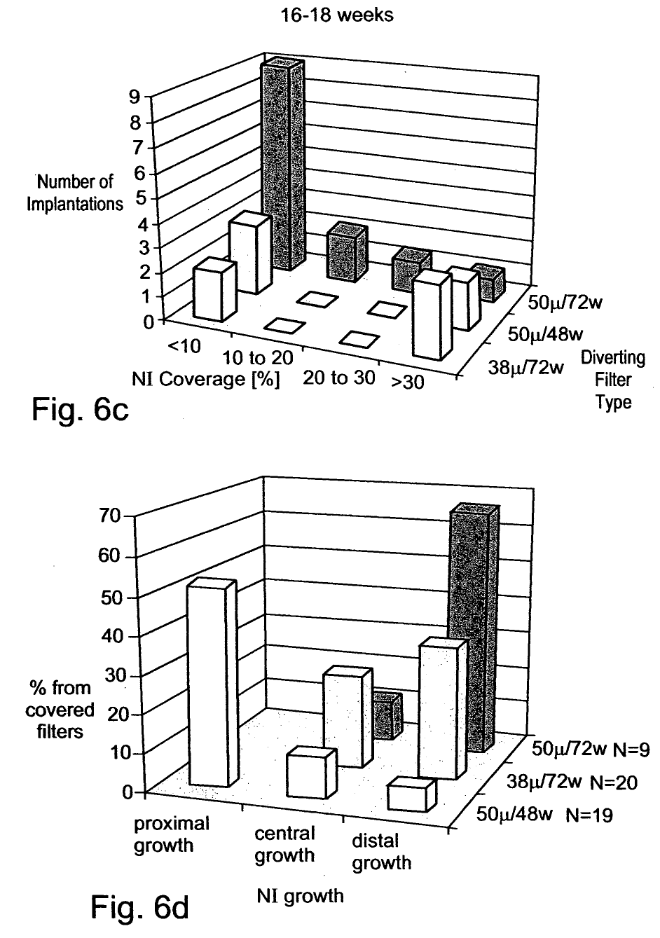

Since NIG is time-dependent, a distribution of NI coverage by follow up (FU) period was assessed. FIGS. 6a-6c illustrate the results of the FU, in which the x-axis represents the percentage of NI coverage, the y-axis represents the number of implantations and the z-axis represents the diverting filter type at short FU periods of 2-4 weeks, medium FU periods of 10-13 weeks and long FU periods of 16-18 weeks, respectively. FIG. 6a representing short follow up periods of filters harvested at 2-4 weeks, shows little difference in NIG between the three designs. However, at medium and long FU periods of 10-13 and 16-18 weeks, respectively, as illustrated in FIGS. 6b and 6c, respectively, the 50μ/48 w and 50μ/72 w prototypes have significantly less NI coverage on the filtering filaments when compared to the 38μ/72 w design.

It is important to identify the direction of NIG, since the direction may point to its origin. FIG. 6d illustrates the direction of NIG, in which the x-axis represents NIG along the longitudinal axis of diverting filter 100, specifically showing distal NIG (portion 130 of FIG. 2), central NIG (portion 110 of FIG. 2) and proximal NIG (portion 120 of FIG. 2). The y-axis represents the percentage of covered filters, and the z-axis represents the type of diverting filter 100. Proximal growth probably relates to a mechanical failure, while distal growth can be referred to anatomical (e.g. when the distal part is facing the orifice of another bifurcation) and hemodynamical conditions, as well as mechanical failures (e.g. an improper distal edge opening, or too small Dᵢ accompanied by an acute implanted braid angle β (<60°).

In the 50μ/72 w and the 38μ/72 w designs the direction of NIG is from the distal part onto the filter, while the 50μ/48 w design shows proximally originating NIG. Exaggerated NIG (>50% at 4 Mo FU) was observed in the 50μ/48 w design correlated with a traceable mechanical failure (local enhanced shortening) detected in the proximal part, suggesting that this mechanical failure might be the only origin of enhanced NIG in the filtering part of the 50μ/48 w design.

In general, for all three design types tested, good mechanical positioning and opening was associated with a smooth and thin coverage by a neointimal layer. Migration from initial location occurred in about 14% of cases in each design. However, some of the migration was negligible, and did not affect the proper positioning and functioning of diverting filter 100. Four (8.3%) had significant migration with the final result of non-filtration.

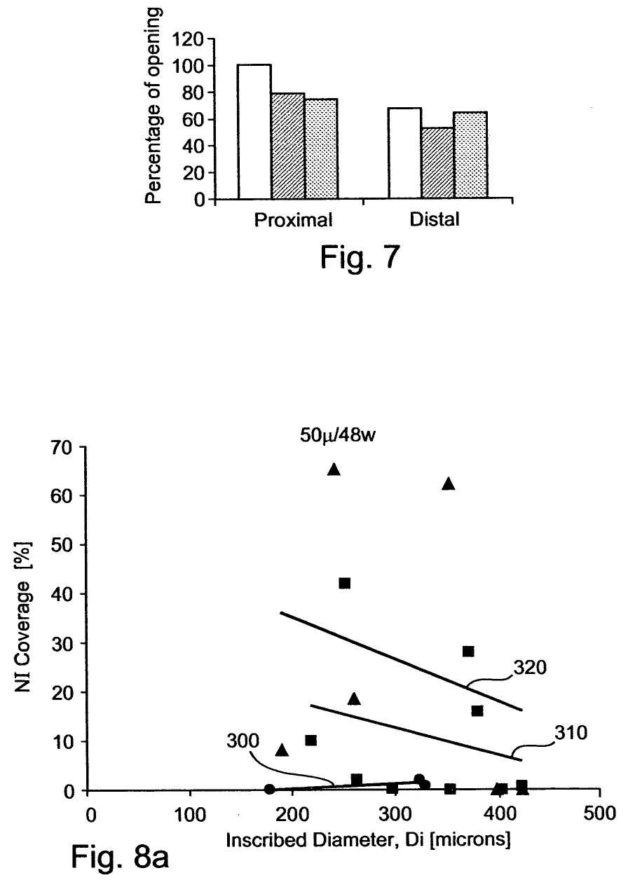

FIG. 7 illustrates an analysis of various mechanical and geometrical parameters versus percentage of NI coverage of the filtering part, in which the x-axis represents proximal and distal opening and the y-axis represents the percentage of samples that fully opened. The 50μ/48 w design, 50μ/72 w design and 38μ/72 design are represented by an open box, a hashed box and a dotted box respectively. For the 50μ/48 w design, an excellent proximal edge opening (100%) with a full tapering to the vessel wall was observed. The distal edge shows some failures (23%) resulting in partial opening, but still well appositioned to the vessel wall. For the 50μ/72 w design, a good proximal edge opening (81%) was observed, but some loose wires were found, so that full compliancy and ultimate adherence to the vessel wall was not achieved. The distal edge showed a poorer adherence (58%). For the 38μ/72 w design, a good proximal edge opening (77%) was observed, however some loose wires were found, which impeded proper adherence to the vessel wall. The distal edges were comparatively less adherent to the vessels wall (67%) than those of the 50μ/48 w design.

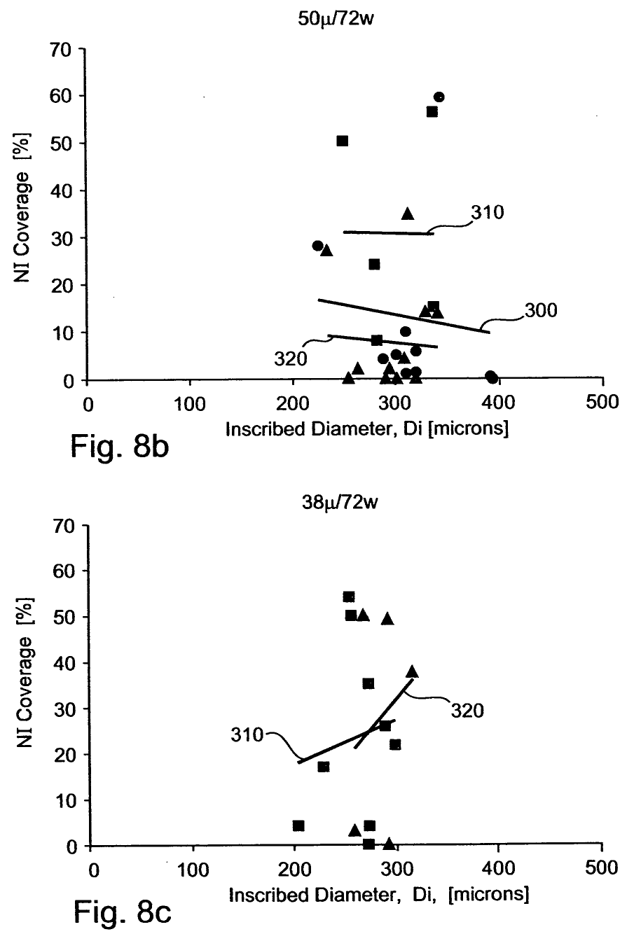

FIGS. 8a-8c illustrate the relationship between Dᵢ and percentage of NI coverage for the 50μ/48 w, 50μ/72 w and 38μ/72 designs, respectively, in which the x-axis represents the inscribed diameter Dᵢ, in microns and the y-axis represents the percentage of NI coverage. Data points at 3 weeks FU are represented by circular marks and a best fit line 300, data points at 9 weeks FU are represented by square marks and a best fit line 310, and data points at 18 weeks FU are represented by triangular marks with a best fit line 320, respectively. Inscribed diameter Dᵢ seems to have the most decisive impact on the 50μ/48 w design as illustrated in FIG. 8a: one can notice that the larger the window the less the NI coverage percentage and thus the higher the patency. The 50μ/72 w design as illustrated in FIG. 8b shows the same pattern, though in a less marked way. It seems that full patency can be achieved for a design comprising 50 μm filaments provided the inscribed diameter is >400μ. However, the 38μ/72 w design as illustrated in FIG. 8c does not present any correlation between the NIG and Dᵢ.

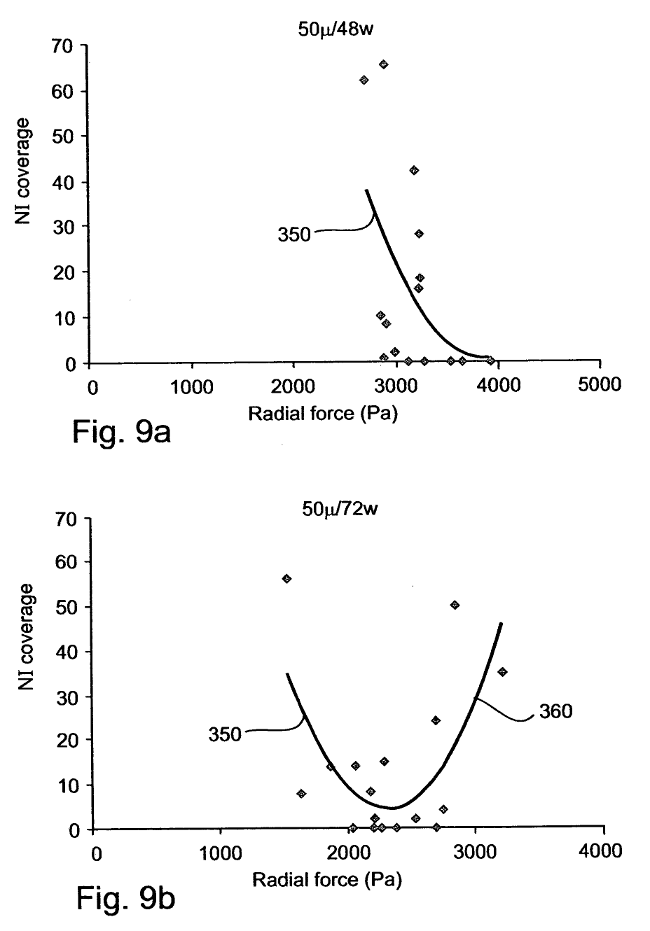

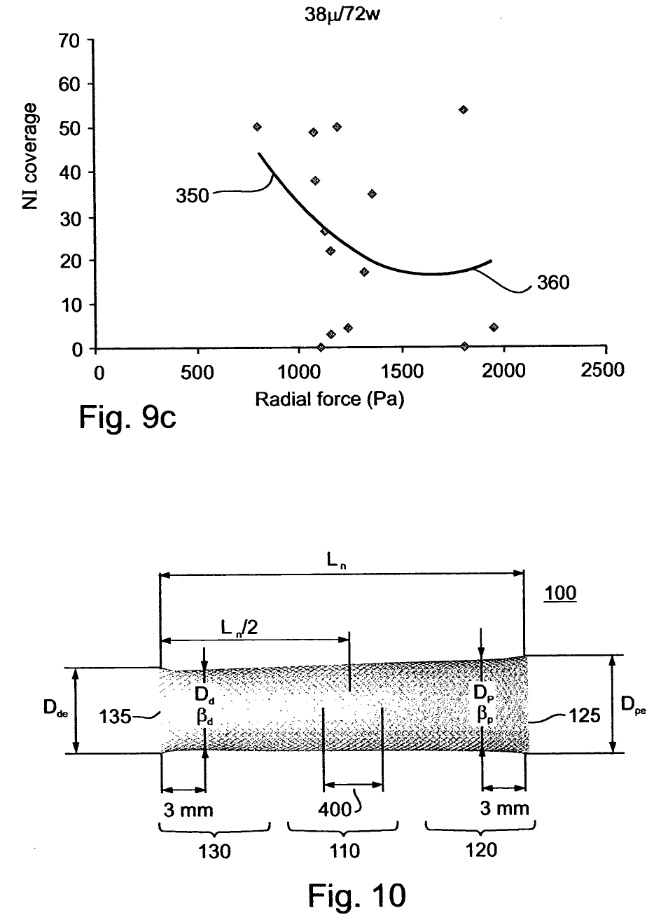

There is thus suggested by the above initial experimentation an optimal region of values for the radial force and inscribed diameter, which in turn, depends on implanted braid angle β. The three tested designs show a relationship when tested for dependency between radial force and NIG. FIGS. 9a-9c illustrate NI coverage as a function of radial force, with the x-axis representing radial force in Pascal and the y-axis representing NI coverage in percentage for the for the 50μ/48 w, 50μ/72 w and 38μ/72 w designs, respectively. The minimal NI coverage point correlates with the ˜90° implanted braid angle β. Left descending portion 350 of the curve shown in FIGS. 9a-9c represent a correlation between radial force and NI coverage, in which the higher the radial force, the lower the NI coverage. Right ascending portion 360 of FIGS. 9b-9c shows an inverse dependency in which increasing radial force corresponds with increased NI coverage, however this is most probably correlated to decreasing Dᵢ and implanted braid angle β. The 50μ/48 w and 50μ/72 w designs, illustrated in FIGS. 9a and 9b respectively, exhibit an equilibrium point with NI coverage <10%. The 38μ/72 w design illustrated in FIG. 9c exhibits a minimum of 20% of NI coverage, since it is both a weak structure, and is characterized by a relatively small Dᵢ(≈300μ). In addition, the radial forces at the equilibrium point of the 50μ/48 w and the 50μ/72 w designs illustrated in FIGS. 9a and 9b, respectively, are significantly higher than the radial force of the 38μ/72 w design illustrated in FIG. 9c, thus providing an additional force to prevent initial migration, and improving the opening and tapering characteristics of the diverting filter.

The above results show that a 50μ/48 w design exhibits proper mechanical behavior (e.g. positioning, opening) of the implants, systematically accompanied by good patency, or maintained blood flow through the vessels. Microscopical observations of the extracted specimens showed a smooth and homogeneous neointimal layer on the stented parts and a thin endothelial layer on the filaments of the filtering part. The 38μ/72 w design was less compatible in terms of vessel patency, mainly because of mechanical failures, such as bad tapering and edge opening. Still, similarly to the 50μ/48 w designs, the stented parts are covered with a thin layer of tissue. The 50μ/72 w design exhibited high neointimal coverage of the filtering parts, primarily due to improper distal opening. However, at 4-month FU in case a good mechanical behavior was observed, clean filters were found with a thin endothelial layer covering the filtering filaments.

Furthermore, the 50μ/48 w design has the largest filtering window size with a Dᵢ>400μ, thus ensuring good patency, and generally exhibited good distal and proximal edge openings. The 50μ/48 w design had a significantly high scoring in a performed angiographical flow evaluation. This design further exhibits a very low percentage of endothelial monolayer covering of the filaments at the filtering part (110 of FIG. 2). Based on the above, the 50μ/48 w is considered to exhibit the best biological and mechanical performance, in particular a design exhibiting PI>=80%, Dᵢ>400 μm comprising round filament wires on the order of 50 μm, and can be considered significantly more favorable compared to other designs.

The clinically relevant nominal diameters for a carotid implant are 7-10 mm. Based on the biological compatibility of the tested 50μ/48 w design having nominal diameters of 7 and 8 mm on a braiding mandrel, similar design characteristics including PI, radial force and filtering window dimensions should be considered for larger diameters. Specific diverting filter specifications were developed based on the above studies, which represent novel improvements over the prior art.

### Tubular 8 mm Specifications

FIG. 10 illustrates a diverting filter 100 designed in accordance with the principle of the current invention. Diverting filter 100 comprises filaments of fine wire braided together to form a self expanding diverting filter having a middle region 110, a proximal region 120 and a distal region 130. Diverting filter 100 is shown at rest, being neither compressed nor expanded, and exhibits a length, Lₙ, as the longitudinal distance between end 125 of proximal region 120 and end 135 of distal region 130. A uniformity detection region 400 defined as the length of 12 window edges or diagonals located within middle region 110, with the center of uniformity detection region 400 being located at Lₙ/2 from distal end 135. Uniformity detection region 400 is used to measure values of the middle region 110, as will be described further hereinto below. It is to be understood that a portion of middle region 110 acts as the diverting filter as shown in FIG. 2, in which a portion of middle region 110 covers the orifice of ICA 30, thus preventing emboli appearing from CCA 20 from entering ICA 30. Diameter Dₚ of proximal region 120 and braid angle βₚ of proximal region 120 are defined at a point 3 mm distally from proximal end 125. Diameter D_{d} of distal region 130 and braid angle β_{d} of distal region 130 are defined at a point 3 mm proximally from distal end 135. Proximal flare Fₚ is defined as the difference between diameter Dₚₑ of proximal end 125 and diameter Dₚ. Distal flare F_{d} is defined as difference between diameter D_{de} of distal end 135 and diameter D_{d}.

Table VII contains a list of parameters and values for the 8mm model of diverting filter 100 in accordance with the principle of the current invention. Diverting filter 100 is defined at two lengths, 65 mm and 85 mm, with the lengths being determined, for convenience, on a delivery system having an outer sheath (142 of FIGS. 3a-3b) exhibiting an inner diameter of 1.7 mm. Average implanted braid angle β of middle region 110 is based on a calculated diameter of (Dₚ+D_{d})/2, due the difficulty of actual measurement.

TABLE VII

| PARAMETER | VALUE |
|---|---|
| Wire Diameter | 50 μm ± 2 |
| Number of Wires | 48 |
| Estimated Implanted Length | 43-54 mm and 56-71 mm ± 2 mm |
| Diameter-mounted in the | 1.7 mm |
| delivery system | |
| Length-mounted in the delivery | 63 to 66 mm and 83-86 mm ± 2 mm |
| system | |
| Length at rest - Lₙ | 26 mm and 32 mm ± 2 mm |
| Proximal Over Sizing | 1.5 to 3.0 mm |
| Average Proximal Radial | >1,500 Pa |
| Pressure | |
| Average Distal Radial Pressure | >900 Pa |
| ECA/CCA Ratio | 0.6 to 1 |
| Average Implanted Braid Angle | 70° to 110° preferably 70° to 100°, and |
| β of Middle Region 110 | further preferably 80° to 100°. |
| Average Implanted Braid Angle | >48°, preferably >60° |
| β_{d} of Distal Region 130 | |
| Average Implanted Braid Angle | <110°, preferably, <100° |
| βₚ of Proximal Region 120 | |
| Dᵢ, Inscribed Diameter of | 400-500 μm preferably 450-500 μm, |
| Implanted Middle region 110 | further preferably 470-500 μm. |
| Average Implanted Porosity | >=80%, preferably 80% to 83% |
| Index - Middle region 110 | |
| Implanted Porosity Index - | >=79%, preferably 80% to 83% |
| Distal Region 130 | |
| Implanted Porosity Index - | >=80%, preferably 80% to 83% |
| Proximal Region 120 | |
| Radiopacity | Radiopaque marker at each end |

Table VIII below contains a list of parameters and preferred values and typical variation for the 8 mm diverting filter 100 in accordance with the principle of the current invention. The term 8 mm is based on a preferred braiding mandrel having a nominal outside diameter of 8 mm with a tolerance of ±0.05 mm.

It is to be understood that the values have a large tolerance because diverting filter 100 is not a completely rigid body. It can be compressed and stretched along its longitudinal axis, which leads to considerable changes in all geometrical values described above. Due to this elastic features it is stabilized in the equilibrium state geometry when released (defined as fully open, at rest or nominal state, the terms being used interchangeably). The equilibrium state has a hysteresis character: it depends on the condition before releasing (compressed or stretched), and is caused by the friction between the filaments.

TABLE VIII

| PARAMETERS | VALUES |
|---|---|
| D_{de} | 8.2 ± 0.4 mm |
| D_{d} | 7.5 ± 0.2 mm |
| F_{d} | >0.4 mm |
| β_{d} | 124 ± 5° |
| Dₚₑ | 8.3 ± 0.2 mm |
| Dₚ | 7.9 ± 0.2 mm |
| Fₚ | >0.2 mm |
| βₚ | 141 ± 5° |

Preferably, distal flaring is obtained by cutting the braid defining diverting filter 100 in the region of a sharp diameter change of the braid, representing a 2-4 window length. Since it is necessary to cut the diverging filter at a precise location within a window, and the window orientation within the expansion region is random, there are large variations in the location of the cutting line. This causes large variations in D_{de}, and thus in F_{d}. However, the design specifications provide the minimum distal flare of 0.40 mm to ensure good distal compliance.

Non-uniformity is specified as up to 15% and is defined as a relative difference between the window size within the uniformity detection region 400 as:

Non Uniformity=Max {(D_{i avg}−D_{i min})/D_{i avg}, (D_{i max}−D_{i avg})/D_{i avg}} Equation 13

where D_{i max} is the maximum inscribed diameter within the uniformity detecting region; D_{i min} is the minimum inscribed diameter within the uniformity detecting region and D_{i avg} is the average inscribed diameter within the uniformity detecting region.

Proximal over sizing is defined as the difference between Dₚ and the diameter of CCA 20 at the implanted location. Preferably, angle 50 formed by the longitudingal axes of CCA 20 and ECA 40 should not exceed 45°.

Average porosity index is the calculated PI at (Dₚ+D_{d})/2 of the implanted diverting filter 100, in which Dₚ and D_{d} are constrained by the artery in which diverting filter 100 is implanted.

### Tubular 9 mm Specifications

Table IX contains a list of parameters and values for the 9 mm diverting filter 100 in accordance with the principle of the current invention. Diverting filter 100 is defined at two lengths, 65 mm and 85 mm, with the lengths being determined, for convenience, on a delivery system having an outer sheath (142 of FIGS. 3a-3b) exhibiting an inner diameter of 1.7 mm. Average implanted braid angle β of middle region 110 is based on a calculated diameter of (Dₚ+D_{d})/2, due the difficulty of actual measurement.

TABLE IX

| PARAMETER | VALUE |
|---|---|
| Wire Diameter | 50 μm ± 2 |
| Number of Wires | 56 |
| Estimated Implanted Length | 43-54 mm and 56-71 mm ± 2 mm |
| Diameter-mounted in the | 1.7 mm |
| delivery system | |
| Length-mounted in the delivery | 63 to 66 mm and 83-86 mm ± 2 mm |
| system | |
| Length at rest - Lₙ | 26 mm and 32 mm ± 2 mm |
| Proximal Over Sizing | 1.5 to 3.0 mm |
| Average Proximal Radial | >900 Pa |
| Pressure | |
| Average Distal Radial Pressure | >700 Pa |
| ECA/CCA Ratio | 0.6 to 1 |
| Average Implanted Braid Angle | 70° to 110° preferably 70° to 100°, and |
| β of Middle Region 110 | further preferably 80° to 100°. |
| Average Implanted Braid Angle | >48°, preferably >60° |
| β_{d} of Distal Region 130 | |
| Average Implanted Braid Angle | <110°, preferably, <100° |
| βₚ of Proximal Region 120 | |
| Dᵢ, Inscribed Implanted | 400-500 μm, preferably 450-500 μm, |
| Diameter of Middle region 110 | further preferably 470-500 μm. |
| Average Implanted Porosity | >=80%, preferably 80% to 83% |
| Index - Middle region 110 | |
| Implanted Porosity Index - | >=79%, preferably 80% to 83% |
| Distal Region 130 | |
| Implanted Porosity Index - | >=80%, preferably 80% to 83% |
| Proximal Region 120 | |
| Radiopacity | Radiopaque marker at each end |

Table X below contains a list of parameters and preferred values and typical variation for the 9 mm diverting filter 100 in accordance with the principle of the current invention. The term 9 mm is based on a preferred braiding mandrel having a nominal outside diameter of 9 mm with a tolerance of ±0.05 mm.

TABLE X

| PARAMETERS | VALUES |
|---|---|
| D_{de} | 8.7 ± 0.4 mm |
| D_{d} | 8.0 ± 0.2 mm |
| F_{d} | >0.4 mm |
| β_{d} | 115 ± 5° |
| Dₚₑ | 9.1 ± 0.2 mm |
| Dₚ | 8.7 ± 0.2 mm |
| Fₚ | >0.2 mm |
| βₚ | 135 ± 5° |

Non-uniformity is specified as up to 15% and is defined in Equation 13 above.

Thus the present embodiments enable a diverting filter for implantation in the bifurcation of the human CCA with the ECA and the ICA having specific design characteristics that will not be occluded in the patient body by emboli or neointimal growth and providing an average PI of at least 80% in the diverting filter region.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and subcombinations of the various features described hereinabove as well as variations and modifications thereof which would occur to persons skilled in the art upon reading the foregoing description.

What is claimed is:

1. A diverting filter for implantation in the bifurcation of the human common carotid artery (CCA) with the external carotid artery (ECA) and the internal carotid artery (ICA), comprising:
a tubular body expandable from an initial small-diameter state for manipulation through the CCA to an expanded larger-diameter state for implantation in said bifurcation;
said tubular body including a proximal region for implantation in the CCA, a distal region for implantation in the ECA, and a middle filtering region for alignment with the orifice of the ICA for diverting relatively-large emboli in the CCA blood flow to the ECA while minimizing interference to blood flow through both the ICA and the ECA;
said tubular body being constituted of between 48 and 56 braided filaments each having an outer diameter of 48-52 um and braided into a tubular body exhibiting an average porosity index of at least 80% when in said expanded state.

2. The diverting filter according to claim 1, wherein said average porosity index is 80-83%.

3. The diverting filter according to claim 1, wherein said tubular body is constituted of one of 48 and 56 of said braided filaments.

4. The diverting filter according to claim 1, wherein said average porosity index in said middle region is defined by windows having an inscribed diameter of 400-500 μm.

5. The diverting filter according to claim 1, wherein said average porosity index in said middle region is defined by windows having an inscribed diameter of 450-500 μm.

6. The diverting filter according to claim 1, wherein said tubular body further exhibits an at rest state wherein said tubular body exhibits a diameter greater than said expanded larger-diameter state, and
wherein in said at rest state of the tubular body said distal region has an outer diameter gradually decreasing from said middle filtering region and terminating in an outwardly flared distal end, and
said proximal region has an outer diameter gradually increasing from said middle filtering region and terminating in an outwardly flared proximal end.

7. The diverting filter according to claim 6, wherein the outer diameter of the outwardly flared distal end is increased by more than 0.4 mm in respect to said distal region in said at rest state.

8. The diverting filter according to claim 6, wherein the outer diameter of the outwardly flared proximal end is increased by more than 0.2 mm in respect to said proximal region in said at rest state.

9. The diverting filter according to claim 6, wherein, in said at rest state of the tubular body, the outer diameter of said distal region is 7.3-7.7 mm.

10. The diverting filter according to claim 6, wherein, in said at rest state of the tubular body, the outer diameter of an end of said distal region is 7.8-8.6 mm.

11. The diverting filter according to claim 6, wherein, in said at rest state of the tubular body, the outer diameter of said proximal region is 7.7-8.1 mm.

12. The diverting filter according to claim 6, wherein, in said at rest state of the tubular body, the outer diameter of an end of said proximal region is 8.1-8.5 mm.

13. The diverting filter according to claim 6, wherein in said at rest state the outer diameter of the outwardly flared distal end is increased by more than 0.4 mm, and the outer diameter of the outwardly flared proximal end is increased by more than 0.2 mm.

14. The diverting filter according to claim 6, wherein the length of said tubular body in said at rest state is 30-34 mm.

15. A diverting filter for implantation in the bifurcation of the human common carotid artery (CCA) with the external carotid artery (ECA) and the internal carotid artery (ICA), comprising:
a tubular body expandable from an initial small-diameter state for manipulation through the CCA to an expanded larger-diameter state for implantation in said bifurcation;
said tubular body including a proximal region for implantation in the CCA, a distal region for implantation in the ECA, and a middle filtering region for alignment with the orifice of the ICA for diverting relatively-large emboli in the CCA blood flow to the ECA while minimizing interference to blood flow through both the ICA and the ECA;
said tubular body being constituted of a plurality of braided filaments each having an outer diameter of 48-52 um and braided into a tubular body exhibiting an average implanted braid angle of 70°-110° in said middle filtering region and an average porosity index of at least 80% when in said expanded state.

16. The diverting filter according to claim 15, having an average implanted braid angle of 70°-105° in said middle filtering region when in said expanded state.

17. The diverting filter according to claim 15, having an average implanted braid angle of 80°-100° in said middle filtering region when in said expanded state.

18. The diverting filter according to claim 15, wherein said plurality of braided filaments is between 48 and 56 braided filaments.

19. The diverting filter according to claim 15, wherein said plurality of braided filaments is one of 48 and 56 braided filaments.

20. A diverting filter for implantation in the bifurcation of the human common carotid artery (CCA) with the external carotid artery (ECA) and the internal carotid artery (ICA), comprising:
a tubular body expandable from an initial small-diameter state for manipulation through the CCA to an expanded larger-diameter state for implantation in said bifurcation, said tubular body having an at rest state wherein said tubular body exhibits a diameter greater than said expanded larger-diameter state;
said tubular body including a proximal region for implantation in the CCA, a distal region for implantation in the ECA, and a middle filtering region for alignment with the orifice of the ICA for diverting relatively-large emboli in the CCA blood flow to the ECA while minimizing interference to blood flow through both the ICA and the ECA;
said tubular body being constituted of a plurality of braided filaments braided into a tubular body exhibiting an inscribed diameter of 400-500 μm in said middle filtering region and an average porosity index of at least 80% when in said expanded state.

21. The diverting filter according to claim 20, having an inscribed diameter of 450-500μ in said middle filtering region when in said expanded state.

22. The diverting filter according to claim 20, wherein said middle filtering region exhibits an average implanted braid angle of 75°-105° in said middle filtering region when in said expanded state.

23. The diverting filter according to claim 20, wherein said plurality of braided filaments is between 48 and 56 braided filaments.

24. The diverting filter according to claim 20, wherein said plurality of braided filaments constitute filaments each having an outer diameter of between 48-52 um.

25. The diverting filter according to claim 23, wherein said plurality of braided filaments constitute filaments each having an outer diameter of between 48-52 um.
